(19) United States
(12) Patent Application Publication
(10) Pub. No.: US 2017/0305842 A1
(43) Pub. Date: Oct. 26, 2017

(54) METHOD FOR PRODUCING ISOCYANATES IN THE GAS PHASE

(71) Applicant: Covestro Deutschland AG, Leverkusen (DE)

(72) Inventors: Josef Sanders, Leverkusen (DE); Armin Schymura, Neuss (DE); Martin Ehrig, Leverkusen (DE); Manfred Keller-Killewald, Dormagen (DE); Dietmar Wastian, Dormagen (DE)

(73) Assignee: Covestro Deutschland AG, Leverkusen (DE)

(21) Appl. No.: 15/512,218

(22) PCT Filed: Sep. 18, 2015
(86) PCT No.: PCT/EP2015/071436

§ 371 Date: Mar. 17, 2017

(30) Foreign Application Priority Data
Sep. 19, 2014 (EP) 14185562.7
Jun. 12, 2015 (EP) 15171773.3

(51) Int. Cl. C07C 263/10

(52) U.S. Cl. CPC C07C 263/10

(57) ABSTRACT

The invention relates to a method for producing isocyanates by reacting the corresponding primary amines with phosgene in the gas phase, wherein the phosgene is injected into the gaseous amine flow in the interior of the flow tube via an outer ring channel and through several radial channels in an angle of ≦90° relative to the flow direction of the gaseous amine flow, wherein a static mixer of the KENICS type is provided coaxially in the interior of the flow tube.

### CROSS-REFERENCE TO RELATED APPLICATIONS

This application is a National Phase Application of PCT/EP2015/071436, filed Sep. 18, 2015, which claims priority to European Application No. 14185562.7, filed Sep. 19, 2014 and European Application No. 15171773.3, filed Jun. 12, 2015, all of which are incorporated herein by reference.

### FIELD OF THE INVENTION

The invention relates to a process for preparing isocyanates by reacting the corresponding primary amines with phosgene in the gas phase.

### BACKGROUND OF THE INVENTION

Isocyanates are produced in large volumes and serve mainly as starting materials for production of polyurethanes. They are usually prepared by reacting the corresponding amines with phosgene. One means of preparation of isocyanates is the reaction of the amines with the phosgene in the gas phase.

It is known that, in gas phase reactions, good mixing of the reactants plays an important role in the achievement of high conversions and selectivities, particularly in the conversion of polyfunctional reactants. The methods of achieving short mixing times are known in principle. Suitable mixing units are those having dynamic or static mixing elements. Preference is given to using static mixers.

Processes where dynamic mixing elements, for example stirrers, are used are described, for example, in patent applications GB 1 165 831 A1 and EP 2 199 277 B1.

For the construction of static mixing elements, a number of different possible implementations are conceivable, for example the use of nozzles known from combustion technology, smooth jet nozzles or Venturi nozzles.

The prior art discloses, in particular, continuous processes for gas phase phosgenation of amines using tubular reactors, where the reactants are mixed by the jet mixer principle using coaxial nozzles, especially smooth jet nozzles (see, for example, Chem.-Ing.-Techn. 44, 1972, p. 1051 ff.). Such processes are described, for example, in patent applications EP 0 289 840 A1, EP 0 570 799 A1, EP 0 699 657 A1, EP 1 275 639 A1, EP 1 319 655 A2, EP 362 847 A2 and EP 2 199 277 B1.

In addition, various patent applications describe variants by which mixing by means of coaxial nozzles can be further improved. For example, EP 1 449 826 B1 discloses a process in which mixing of the vaporous reactants is accomplished using multiple nozzles parallel to the flow direction.

EP 1 526 129 A1 discloses a process in which flow turbulence in the mixing zone is increased by means of internals, for example by means of swirl spirals.

EP 1 555 258 A1 discloses a process using an annular gap nozzle, in which the gaseous amine, optionally diluted with an inert gas, is supplied to the reactor via the annular gap, and the phosgene is supplied via the inner nozzle as a central jet and over the remaining reactor cross section.

EP 188 247 A1 (WO 2009/027232 A1) and EP 2 188 248 A1 (WO 2009/027234 A1) describe processes in which an inert medium, for example nitrogen, is metered in between the two fluid streams of amine and phosgene in a mixing element, for example in a three-phase mixing nozzle. According to the teaching of EP 2 188 248 A1, the turbulent flow interface of at least one fluid stream is additionally reduced by at least one mechanical baffle prior to contacting with the other stream.

Even though coaxial mixing of amines with phosgene by means of nozzles has now become established in industry, the prior art also describes alternative mixing elements.

EP 0 928 785 A1 discloses a process in which rapid mixing of the fluid reactant streams is accomplished using microstructure mixers. However, it is disadvantageous here that, because of the small dimensions of the mixers, blockages resulting from deposition of solid by-products or breakdown products occur very easily at the high temperatures, which is the reason why this process has not become widely established on the industrial scale.

EP 2 088 139 A1 describes a process in which gaseous amine is injected at 200-600° C. from an outer annular channel through radial holes at right angles into excess phosgene flowing in a flow tube. After the mixing, the reactant stream is first accelerated by a reduction in cross section and, shortly thereafter, slowed down again by an increase in cross section, before it is then guided into a reactor.

WO 2011/115848 A1 discloses a static mixer and the use thereof for preparation of isocyanates by phosgenation of primary amines, wherein an amine stream containing up to 90% solvent is injected at right angles via an outer annular channel through a plurality of radial holes into the phosgene stream in the interior of a flow tube, wherein a (very short) flow element in the flow tube at the level of the holes imparts an annular flow to the phosgene stream without any change in the flow rate, which promotes mixing with the amine stream. However, no examples of isocyanate preparation are cited, and there is likewise a lack of any volume, pressure and temperature figures, and so no conclusions about the state of matter of the reactants are possible. The only amine mentioned is MDA.

Even though some of the processes described have now been implemented on the industrial scale, they still have some disadvantages. Particular mention should be made here of the shortening of the lifetimes of the plants (plant lifetimes) by soiling caused by the formation of solid by-products.

In the processes using moving mixing elements, for example stirrers, mixing is not rapid enough in spite of the high stirrer speeds employed, which leads to a broad contact time distribution and hence to unwanted formation of solids. The sealing of their driveshafts at the point where they enter the reactor, which is necessary from a safety point of view, is also difficult and entails a high level of testing and maintenance.

Disadvantages of the processes based on the jet mixer principle are, for example, high pressure drop or insufficiently rapid mixing. A high pressure drop in the mixing element causes elevated complexity in the supply of the gaseous reactants and requires higher boiling temperatures in order to assure adequate supply pressure. Particularly in the case of reactants having reactive functional groups, the elevated boiling temperature, however, results in thermal damage and hence increased formation of by-products (yield/selectivity losses). Insufficiently rapid mixing or backmixing additionally lead to elevated dwell time of a portion of the reactants and products and, as a result, to unwanted parallel or further reactions. Moreover, inadequate mixing, particularly in the case of strongly exothermic or endothermic reactions, causes an inhomogeneous temperature distribution in the reactor. Such “hot spots” or “cold spots” in the reactor lead to increased thermal breakdown of the products or to unwanted premature condensation of the products. Thermal breakdown products form solid residue which is deposited at the reactor wall. Sufficiently rapid mixing requires high flow rates at which the dwell times necessary for full conversion of the amines, specifically in the case of use of aromatic amines, can be achieved only in very long mixing and reactor tubes. Further disadvantages are the high costs of the mixing elements, which are costly to construct and difficult to manufacture, and the requirement for exact positioning thereof in the center of the reactor cross section. Furthermore, these mixing elements have a tendency to oscillate or to bend during operation, which leads to asymmetry of the flow and hence to rapid soiling of the reactor.

The processes described in EP-A 2 088 139 A1 and WO 2011/115848 A1, in which the amine stream is injected into the phosgene stream in the interior of a flow tube at right angles via an outer annular channel through a plurality of radial holes, have a high pressure drop on the amine side with the disadvantages described above.

### SUMMARY OF THE INVENTION

The present invention provides a process for preparing isocyanates by reacting the corresponding primary amines with phosgene in the gas phase, which avoids the described disadvantages of the known processes and with which high yields and simultaneously long plant lifetimes can be achieved.

These and other advantages and benefits of the present invention will be apparent from the Detailed Description of the Invention herein below.3

### BRIEF DESCRIPTION OF THE FIGURES

The present invention will now be described for purposes of illustration and not limitation in conjunction with the figures, wherein:

FIG. 1 shows a schematic construction of the mixing unit of the invention;

### DETAILED DESCRIPTION OF THE INVENTION

It has been found that, surprisingly, primary amines can be reacted with phosgene in a tubular reactor above the boiling temperature of the amine in the gas phase advantageously with long plant lifetimes when the phosgene is injected into the gaseous amine stream in the interior of a flow tube at an angle of ≦90° relative to the flow direction of the gaseous amine stream via an outer annular channel through a plurality of radial channels, with a static mixer of the KENICS type present in a coaxial arrangement in the interior of the flow tube. Mixers of this kind are also referred to in the literature as gas mixer, helical chain or V element mixers.

The invention provides a process for preparing di- or triisocyanates of the general formula (I) or (II)

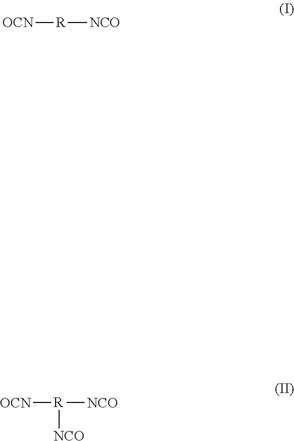

or mixtures of such di- and/or triisocyanates

where

R is a (cyclo)aliphatic, araliphatic or aromatic hydrocarbyl radical having up to 15 carbon atoms, with the proviso that at least 2 carbon atoms are arranged between the NCO groups,

by phosgenating the corresponding di- and/or triamines of the general formula (III) or (IV)

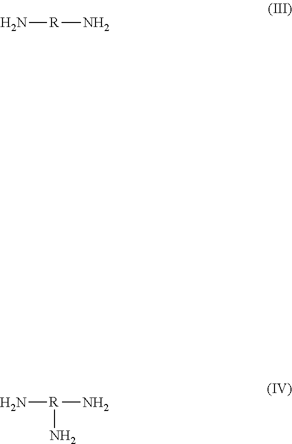

in which

R is as defined above,

in which the vaporous di- and triamines, optionally diluted with an inert gas or with the vapors of an inert solvent, and phosgene are heated separately to temperatures of 200° C. to 600° C., mixed in a tubular reactor and reacted, characterized in that the phosgene is injected into the gaseous amine stream in the interior of a flow tube at an angle of ≦90° relative to the flow direction of the gaseous amine stream via an outer annular channel through a plurality of radial channels, with a static mixer of the KENICS type present in a coaxial arrangement in the interior of the flow tube.

Vaporous amines are understood in accordance with the invention to mean di- and triamines which are in gaseous form and may optionally contain fractions of unevaporated amine droplets (aerosol). Preferably, however, the vaporous amines do not contain any droplets of unevaporated amines

In a first preferred embodiment, the angle is ≧60° and ≦90°, preferably ≧75° and ≦90° and more preferably 90°. An angle of 90° is also referred to in accordance with the invention as “at right angles” or “perpendicular”.

Channels are understood in accordance with the invention to mean orifices which are permeable with respect to the reactant streams. The orifices may have any desired shape and can be obtained, for example, with the aid of a laser or a drill. Preference is given here to holes.

In a particularly preferred embodiment, the channels are holes and the angle is 90°, such that the phosgene is injected into the gaseous amine stream in the interior of a flow tube at right angles via an outer annular channel through a plurality of radial holes.

Typical examples of suitable aliphatic amines are given, for example, in EP 0 289 840 A1. Preference is given to using diamines such as the pure isomers or isomer mixtures of isophoronediamine (IPDA, isomer mixture), hexamethylene-1,6-diamine (HDA), bis(p-aminocyclohexyl)methane, 1,3- or 1,4-bis(aminomethyl)cyclohexane or isomer mixtures thereof, xylylenediamine or isomer mixtures thereof, and more preferably pentane-1,5-diamine. A preferred triamine used is 1,8-diamino-4-(aminomethyl)octane (triaminononane).

Typical examples of suitable aromatic amines are the pure isomers or isomer mixtures of diaminobenzene, of diaminotoluene, of diaminodimethylbenzene, of diaminonaphthalene and of diaminodiphenylmethane. Preference is given to using tolylene-2,4/2,6-diamine mixtures of the isomer ratios 80/20 and 65/35 or the pure tolylene-2,4-diamine isomer.

The starting amines of the formula (III) and (IV) are evaporated prior to performance of the process of the invention, optionally diluted with an inert gas such as N₂, He, Ar or with the vapors of an inert solvent, for example aromatic hydrocarbons with or without halogen substitution, heated to 200° C. to 600° C., preferably 250° C. to 450° C., and supplied to the mixer or reactor.

The amount of any inert gas or gaseous solvent used as diluent is uncritical. For example, the volume ratio of amine vapor to inert gas or solvent vapor may be between 1:0.5 and 1:2.

The phosgene used in the phosgenation is used in excess based on the amine. In general, the molar excess of phosgene based on an amino group is 30% to 300%, preferably 60% to 200%. Before being fed into the mixer or reactor, the phosgene is heated to a temperature of 200° C. to 600° C., preferably 250° C. to 450° C.

In one embodiment, the phosgene can be diluted with an inert gas such as N₂, He, Ar or with the vapors of an inert solvent, for example aromatic hydrocarbons with or without halogen substitution. Preference is given to the undiluted variant.

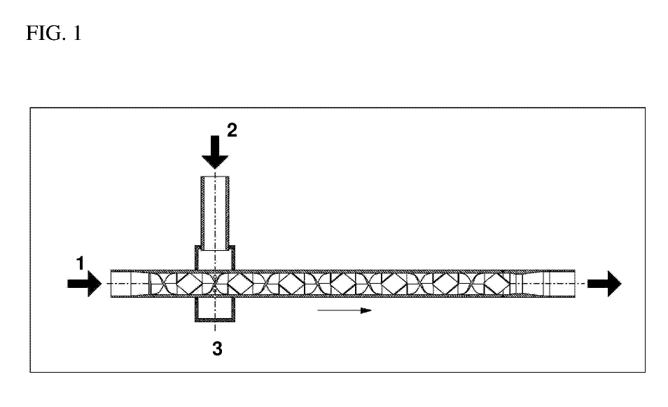

The two reactant streams are mixed in a flow tube in which there is a static mixer of the KENICS type in a coaxial arrangement, wherein the gaseous amine (gas stream A) optionally diluted with an inert gas is guided coaxially through the flow tube and the mixer and the phosgene is introduced at an angle of ≦90°, preferably ≧60° and ≦90°, more preferably ≧75° and ≦90° and most preferably of 90° relative to the flow direction of the amine stream via an outer annular channel through a plurality of radial channels, preferably holes, in a plane at the periphery of the flow tube (plane of introduction). The static mixer already begins above the plane of introduction of the phosgene, in flow direction, and ends in the downstream direction well below the plane of introduction of the phosgene, and its length is dependent on the reactivity of the amine used. In a preferred embodiment of the process of the invention, the number of mixing elements of the static mixer above the plane of introduction is such that any inert gas added to the amine is mixed homogeneously therewith, i.e. the degree of mixing of amine and inert gas immediately upstream of the plane of introduction is 50 to 100, preferably 70 to 95%. The ratio of the length of the static mixer upstream of the plane of introduction to the length beyond the plane of introduction is 0.1 to 1.0, more preferably 0.2 to 0.8. The schematic construction of the mixing unit of the invention is illustrated by way of example in FIG. 1.

The phosgene stream is introduced into the gas stream A at a velocity of 15-90 m/s, more preferably 20-80 m/s. The number of channels and the cross-sectional area thereof are determined from the volume flow rate of phosgene to be introduced. Preference is given to an odd number of channels.

The diameter of the mixing tube is such that the flow rate of the gas mixture of all components directly below (downstream of) the plane of introduction is 4-25 m/s, preferably 6-15 m/s, more preferably 8-12 m/s.

The length of the static mixer of the KENICS type and the number of mixing elements therein are such that a sufficient degree of mixing is achieved at the end of the mixer and can be ascertained by calculation. The design of such mixers is described extensively in the technical literature, for example in “Mischen and Rühren” [Mixing and Stirring], ed.: Matthias Kraume, Wiley-VCH Verlag GmbH & Co. KGaA, Weinheim 2003, p. 198-220, and in the literature cited therein. The degree of mixing to be achieved is dependent on the reactivity of the amine used and is generally 50-100%, preferably 80-99%, more preferably 95-98%.

In the process of the invention, preference is given to using tubular reactors without internals and without moving parts in the interior of the reactor. The tubular reactors generally consist of steel, glass, alloyed or enameled steel, and the dimensions are such that complete reaction of the amine with the phosgene is enabled under the process conditions. The gas streams are introduced into the tubular reactor at one end thereof via a mixing unit described in detail above. The temperature in the reactor is 200° C. to 600° C., preferably 250° C. to 450° C., and this temperature can optionally be maintained by heating the tubular reactor.

In the performance of the process of the invention, in general, the pressure in the inlets to the reaction space is 200-3000 mbar abs., preferably 800-1500 mbar abs., and at the outlet from the reaction space is 150-2000 mbar abs., preferably 750-1440 mbar abs., with observation of a flow rate within the reaction space of 3 to 120 m/s, preferably 5 to 75 m/s, by maintaining a suitable pressure differential. Under these prerequisites, turbulent flow conditions generally exist within the reaction space.

The dwell time is calculated from the throughput of the reactant streams per unit time, the dimensions of the reactor and the reaction parameters of pressure and temperature. According to the reactivity of the amine used, the dwell time of the reaction mixture in the reactor is 0.05 s to 10 s, preferably 0.08 s to 4 s.

On completion of the phosgenation reaction in the reaction space, the gaseous mixture continuously leaving the reaction space is freed of the isocyanate formed. This can be effected, for example, with the aid of an inert solvent, the temperature of which is chosen such that it is, on the one hand, above the breakdown temperature of the carbamoyl chloride corresponding to the isocyanate and, on the other hand, below the condensation temperature of the isocyanate and preferably also of any solvent used as diluent in vaporous form, such that isocyanate and auxiliary solvent condense or the isocyanate dissolves in the auxiliary solvent, while excess phosgene, hydrogen chloride and any inert gas used as diluent pass through the condensation stage or the solvent in gaseous form. Particularly suitable solvents for selectively obtaining the auxiliary solvent from the mixture leaving the reaction space in gaseous form are solvents of the type mentioned by way of example above that are kept at a temperature of 60 to 200° C., preferably 90 to 170° C., especially technical grade monochlorobenzene (MCB) and dichlorobenzene (ODB). Preference is given to MCB. Conceivable methods of selective condensation of isocyanate formed out of the gas mixture leaving the reactor using solvents of this kind are, for example, the passing of the gas mixture through the solvent mentioned or the injection of the solvent (solvent mist) into the gas stream (quench).

The gas mixture that passes through the condensation stage for obtaining the isocyanate is subsequently freed of excess phosgene in a manner known per se. This can be effected by means of a cold trap, absorption in an inert solvent (e.g. MCB or ODB) kept at a temperature of −10° C. to 8° C., or adsorption and hydrolysis on activated carbon. The hydrogen chloride gas that passes through the phosgene recovery stage can be recycled in a manner known per se to recover the chlorine required for phosgene synthesis.

The isocyanate can be purified by fractional distillation or by recrystallization or by adsorptive removal of impurities, for example by treatment with activated carbon, kieselguhr, silica gel or bleaching earth. If the isocyanate has sufficient thermal stability, the purification is preferably effected by very gentle distillative workup of the crude isocyanate solution in the solvent used for isocyanate condensation, with distillation of the isocyanate fraction optionally under reduced pressure.

It is particularly surprising that it is possible with the mixing unit of the invention elucidated in detail above to phosgenate even very reactive amines, for example pentane-1,5-diamine (PDA), with excellent yields and plant lifetimes, to give pentane 1,5-diisocyanate (PDI). The flow rates required are comparatively low, and so it is possible to use shorter reactors than in the case of use of jet mixers. Since the amine, by contrast with the processes described in EP 2 088 139 A1 and WO 2011/115848, is not injected through narrow holes, but is fed to the reactor through a flow tube with a much greater cross section, the pressure drop is very low and hence the evaporation temperature is low. In this way, it is specifically also possible to phosgenate sensitive amines, for example bis(p-aminocyclohexyl)methane or xylylene-1,3-diamine, with better yields.

The invention provides a process for preparing di- or triisocyanates of the general formula (I) or (II)

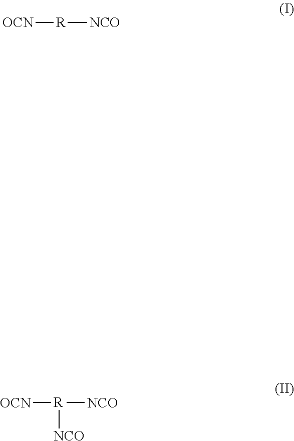

or mixtures of such di- or triisocyanates

where

R is a (cyclo)aliphatic, araliphatic or aromatic hydrocarbyl radical having up to 15 carbon atoms, with the proviso that at least 2 carbon atoms are arranged between the NCO groups,

by phosgenating the corresponding di- or triamines of the general formula (III) or (IV)

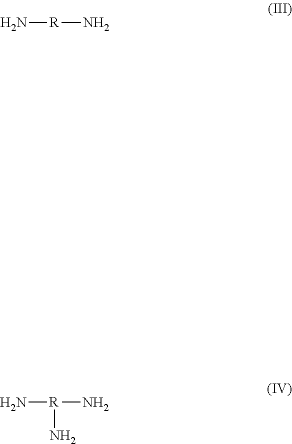

in which

R is as defined above,

in which the vaporous di- and triamines, optionally diluted with an inert gas or with the vapors of an inert solvent, and phosgene are heated separately to temperatures of 200° C. to 600° C., mixed in a tubular reactor and reacted, characterized in that the phosgene is injected into the gaseous amine stream in the interior of a flow tube at an angle of ≦90° relative to the flow direction of the gaseous amine stream via an outer annular channel through a plurality of radial channels, with a static mixer of the KENICS type present in a coaxial arrangement in the interior of the flow tube.

In a second embodiment of the process, the channels are holes.

In a third embodiment of the process according to either of embodiments 1 and 2, the angle is ≧60° and ≦90°.

In a fourth embodiment of the process according to either of embodiments 1 and 2, the angle is ≧75° and ≦90°.

In a fifth embodiment of the process according to either of embodiments 1 and 2, the angle is 90°.

In a sixth embodiment of the process, the process is conducted in such a way that the phosgene is injected into the gaseous amine stream in the interior of a flow tube at right angles via an outer annular channel through a plurality of radial holes.

In a seventh embodiment, the process according to any of embodiments 1 to 6 is conducted in such a way that the amine stream is heated to 250° C. to 450° C. before being fed into the mixer.

In an eighth embodiment, the process according to any of embodiments 1 to 7 is conducted in such a way that the phosgene stream is heated to 250° C. to 450° C. before being fed into the mixer.

In a ninth embodiment, the process according to any of embodiments 1 to 8 is conducted in such a way that the phosgene is used in excess based on the amine and the molar phosgene excess based on an amino group is 30% to 300%.

In a tenth embodiment, the process according to any of embodiments 1 to 8 is conducted in such a way that the phosgene is used in excess based on the amine and the molar phosgene excess based on an amino group is 60% to 200%.

In an eleventh embodiment, the process according to any of embodiments 1 to 10 is conducted in such a way that the phosgene stream is introduced at a velocity of 15-90 m/s into the gaseous amine stream optionally diluted with an inert gas.

In a twelfth embodiment, the process according to any of embodiments 1 to 10 is conducted in such a way that the phosgene stream is introduced at a velocity of 20-80 m/s into the gaseous amine stream optionally diluted with an inert gas.

In a thirteenth embodiment, the process according to any of embodiments 1 to 12 is conducted in such a way that the ratio of the length of the static mixer upstream of the plane of introduction to the length downstream of the plane of introduction FIG. 1 is 0.1 to 1.0.

In a fourteenth embodiment, the process according to any of embodiments 1 to 12 is conducted in such a way that the ratio of the length of the static mixer upstream of the plane of introduction to the length downstream of the plane of introduction FIG. 1 is 0.2 to 0.8.

In a fifteenth embodiment, the process according to any of embodiments 1 to 14 is conducted in such a way that the diameter of the mixing tube is such that the flow rate of the gas mixture of all components immediately below (downstream of) the plane of introduction FIG. 1 is 4-25 m/s.

In a sixteenth embodiment, the process according to any of embodiments 1 to 14 is conducted in such a way that the diameter of the mixing tube is such that the flow rate of the gas mixture of all components immediately below (downstream of) the plane of introduction FIG. 1 is preferably 6-15 m/s.

In a seventeenth embodiment, the process according to any of embodiments 1 to 14 is conducted in such a way that the diameter of the mixing tube is such that the flow rate of the gas mixture of all components immediately below (downstream of) the plane of introduction FIG. 1 is 8-12 m/s.

In an eighteenth embodiment, the process according to any of embodiments 1 to 17 is conducted in such a way that the length of the static mixer of the KENICS type and the number of mixing elements therein is such that a degree of mixing of 50-100% is attained at the end of the mixer.

In a nineteenth embodiment, the process according to any of embodiments 1 to 17 is conducted in such a way that the length of the static mixer of the KENICS type and the number of mixing elements therein is such that a degree of mixing of 80-99% is attained at the end of the mixer.

In a twentieth embodiment, the process according to any of embodiments 1 to 17 is conducted in such a way that the length of the static mixer of the KENICS type and the number of mixing elements therein is such that a degree of mixing of 95-98% is attained at the end of the mixer.

In a twenty-first embodiment, the process according to any of embodiments 1 to 20 is conducted in such a way that an inert gas is added to the amine and the number of mixing elements in the static mixer above the plane of introduction (FIG. 1) is such that the degree of mixing of amine and inert gas immediately upstream of the plane of introduction is 50% to 100%.

In a twenty-second embodiment, the process according to any of embodiments 1 to 20 is conducted in such a way that an inert gas is added to the amine and the number of mixing elements in the static mixer above the plane of introduction FIG. 1 is such that the degree of mixing of amine and inert gas immediately upstream of the plane of introduction is 70% to 95%.

In a twenty-third embodiment, the process according to any of embodiments 1 to 22 is conducted in such a way that the temperature in the reaction space is 200° C. to 600° C.

In a twenty-fourth embodiment, the process according to any of embodiments 1 to 22 is conducted in such a way that the temperature in the reaction space is 250° C. to 450° C.

In a twenty-fifth embodiment, the process according to any of embodiments 1 to 24 is conducted in such a way that the pressure in the inlets to the reaction space is 200-3000 mbar abs. and at the outlet from the reaction space is 150-2000 mbar abs.

In a twenty-sixth embodiment, the process according to any of embodiments 1 to 24 is conducted in such a way that the pressure in the inlets to the reaction space is 800-1500 mbar abs. and at the outlet from the reaction space is 750-1440 mbar abs.

In a twenty-seventh embodiment, the process according to any of embodiments 1 to 26 is conducted in such a way that a flow rate within the reaction space of 3 to 120 m/s is observed.

In a twenty-eighth embodiment, the process according to any of embodiments 1 to 26 is conducted in such a way that a flow rate within the reaction space of 5 to 75 m/s is observed.

In a twenty-ninth embodiment, the process according to any of embodiments 1 to 28 is conducted in such a way that the dwell time of the reaction mixture in the reactor is 0.05 s to 10 s.

In a thirtieth embodiment, the process according to any of embodiments 1 to 28 is conducted in such a way that the dwell time of the reaction mixture in the reactor is 0.08 s to 4 s.

### EXAMPLES

FIG. 1: Schematic construction of a mixing unit of the invention, in which the numbers 1-3 have the following meaning: 1: amine+inert gas; 2: phosgene; 3: plane of introduction.

### GC Method of PDI Analysis:

Gas chromatograph: Agilent (formerly Hewlett PACKARD), 7890, Series A or B (6890 Series A or B are also possible)
Separation column: RXI 17 (Restek), fused silica, length 30 m, internal diameter 0.32 mm, film thickness 1.0
Temperatures: injector 250° C., detector (FID) 350° C.
Oven: Start 80° C., hold time 0 min
   Heating rate 10 K/min to 140° C., hold time 7.5 min.
   Heating rate 20 K/min to 250° C., hold time 5.0 min.
   Run time 24 min
Carrier gas: hydrogen
Gas setting constant flow rate rather than constant pressure
   Column pressure about 0.4 bar abs., at startof analysis
   Column flow about 100 mL/min at constant flow rate
   Split outlet flow rate 100 mL/min
   Ratio of 50:1 Septum purge about 3 mL/min.

### Comparative Example: Phosgenation of Pentane-1,5-Diamine (PDA) with Coaxial Nozzle

In a Miniplant for continuous gas phase phosgenation with an amine evaporation stage, a tubular reactor (length 720 mm, internal diameter 8 mm) having a coaxial nozzle (internal diameter 2 mm) arranged on the reactor axis and a downstream isocyanate condensation stage, at a pressure of 700 mbar abs., measured at the end of the isocyanate condensation stage, 250 g/h of PDA were evaporated continuously with introduction of a nitrogen stream of 37 g/h, superheated to 270° C. and fed to the reactor via the coaxial nozzle (simple smooth jet nozzle). Simultaneously and in parallel thereto, 1090 g/h of phosgene were heated to 300° C. and, in the annular space left clear by the nozzle, likewise fed continuously to the reactor, in which the two reactant streams are mixed and reacted. The velocity of the gas stream in the reactor was about 7.1 m/s and the velocity ratio of the amine/nitrogen stream to the phosgene stream was 5.9. After a mean dwell time in the reactor of 0.1 s, the gas stream containing the pentane 1,5-diisocyanate (PDI) reaction product was cooled down by injection cooling with 5 kg/h of liquid monochlorobenzene and condensed/dissolved, and the temperature of the liquid phase in the quench was about 90° C. The vapors containing MCB and PDI formed were guided together with the offgas from the reaction into an isocyanate absorption column. Isocyanate and MCB were condensed in a downstream cooler and recycled into the quench, while the offgas consisting essentially of nitrogen, excess phosgene and HCl was sent to a phosgene destruction operation. The solution of the isocyanate in MCB obtained was collected and worked up by distillation in portions.

Of about 50 experiments conducted in which the phosgene excess was varied in the range of 125%-175%, the temperature of amine, nitrogen and phosgene was varied in the range of 260-360° C. and the pressure was varied in the range of 0.7-1.3 bar, run times of >4 h were attained only in 3 experiments. The rest of the experiments had to be terminated prematurely, some after only a few minutes, because of rising pressure due to blockages of nozzle and reactor.

Secondary Components in the Phosgenation of PDA:

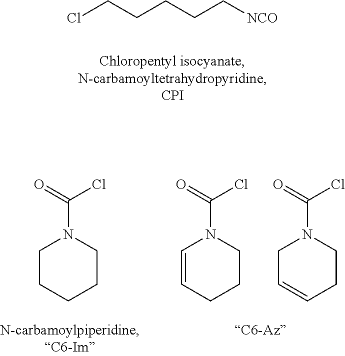

The GC analysis of the crude solution obtained showed the following composition (excluding MCB, area %):

| | |
|---|---|
| CPI | 0.499 |
| C6-Az | 0.195 |
| C6-Im | 3.893 |
| PDI | 95.414 |

### Example 1: Phosgenation of Pentane-1,5-Diamine (PDA) with a Static Mixer (Inventive)

In a Miniplant for continuous gas phase phosgenation with an amine evaporation stage, a tubular reactor (length: 400 mm, internal diameter 8.8 mm) having a mixing tube (length: 210 mm, internal diameter 8 mm) arranged on the reactor axis with a static mixer of the KENICS type (length 200 mm, diameter 7.9 mm, 15 mixing elements) incorporated in a coaxial arrangement therein and a downstream isocyanate condensation stage, at a pressure of 700 mbar abs., measured at the end of the isocyanate condensation stage, 210 g/h of PDA were evaporated continuously with introduction of a nitrogen stream of 37 g/h, superheated to 310° C. and fed to the reactor via the mixing tube. At the same time, 1090 g/h of phosgene were metered into the interior of the mixing tube via an outer annular channel through 7 radial holes (diameter 1 mm) at right angles to the flow direction of the amine. The mean dwell time in the mixer was 0.02 s. The reaction mixture leaving the mixer was fed to the reactor. The mean velocity of the gas stream in the reactor was about 6 m/s and the velocity ratio of the amine/nitrogen stream to the phosgene stream was 0.043. After a mean dwell time in the reactor of 0.12 s, the gas stream containing the PDI reaction product, as described in the comparative example, was quenched with 5 kg/h of liquid MCB and worked up.

With this plant configuration, it was possible to operate the Miniplant without any problem for 80 h.

The GC analysis of the crude solution obtained showed the following composition (excluding MCB, area %):

| | |
|---|---|
| CPI | 0.286 |
| C6-Az | 0.032 |
| C6-Im | 0.004 |
| PDI | 99.678 |

### Example 2: Phosgenation of an Isomer Mixture of Tolylene-2,4- and -2,6-Diamine with a Static Mixer (Inventive)

In a Miniplant for continuous gas phase phosgenation with an amine evaporation stage, a tubular reactor (length: 3100 mm, internal diameter 16.0 mm) having a mixing tube (length: 210 mm, internal diameter 8 mm) arranged on the reactor axis with a static mixer of the KENICS type (length 200 mm, diameter 7.9 mm, 15 mixing elements) incorporated in a coaxial arrangement therein and a downstream isocyanate condensation stage, at a pressure of 1000 mbar abs., measured at the end of the isocyanate condensation stage, 210 g/h of a technical isomer mixture of tolylene-2,4- and -2,6-diamine (TDA) in a ratio of 4:1 are evaporated continuously with introduction of a nitrogen stream of 40 g/h, superheated to 330° C. and fed to the reactor via the mixing tube. At the same time, 1020 g/h of phosgene are metered into the interior of the mixing tube via an outer annular channel through 7 radial holes (diameter 1 mm) at right angles to the flow direction of the amine. The mean dwell time in the mixer is 0.03 s. The reaction mixture leaving the mixer is fed to the reactor. The mean velocity of the gas stream in the reactor is about 1.3 m/s and the velocity ratio of the amine/nitrogen stream to the phosgene stream is 0.04. After a mean dwell time in the reactor of 2.4 s, the gas stream containing the tolylene diisocyanate (TDI) reaction product, analogously to the comparative example, is quenched with 5 kg/h of liquid o-dichlorobenzene (ODB) and worked up.

With this plant configuration, it was possible to operate the Miniplant without any problem for 80 h.

The GC analysis of the crude solution obtained shows the following composition (excluding ODB, area %):

| | |
|---|---|
| Phenylene | 0.016 |
| diisocyanate | |
| Xylylene | 0.007 |
| diisocyanate | |
| TDI | 99.977 |

Various aspects of the subject matter described herein are set out in the following numbered clauses:

1. A process for preparing di- or triisocyanates of the general formula (I) or (II)

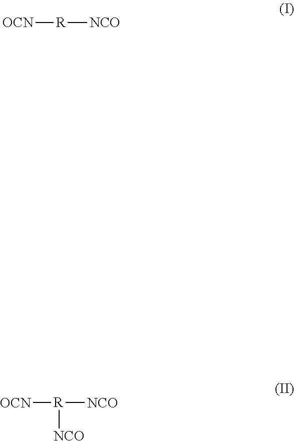

or mixtures of such di- or triisocyanates

where

R is a (cyclo)aliphatic, araliphatic or aromatic hydrocarbyl radical having up to 15 carbon atoms, with the proviso that at least 2 carbon atoms are arranged between the NCO groups,

by phosgenating the corresponding di- and/or triamines of the general formula (III) or (IV)

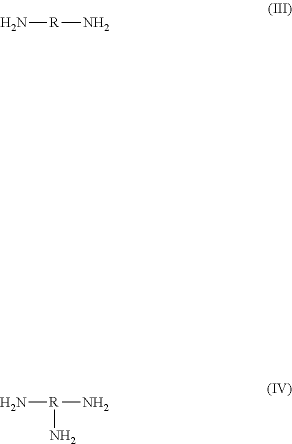

in which

R is as defined above,

in which the vaporous di- and triamines, optionally diluted with an inert gas or with the vapors of an inert solvent, and phosgene are heated separately to temperatures of 200° C. to 600° C., mixed in a tubular reactor and reacted, characterized in that

the phosgene is injected into the gaseous amine stream in the interior of a flow tube at an angle of ≦90° relative to the flow direction of the gaseous amine stream via an outer annular channel through a plurality of radial channels, with a static mixer of the KENICS type present in a coaxial arrangement in the interior of the flow tube.

2. The process as in clause 1, wherein the channels are holes and/or the angle is ≧60° and ≦90°, preferably ≧75° and ≦90° and more preferably 90°.

3. The process as in either of clauses 1 and 2, wherein the di- and/or triamines used are aliphatic amines such as the pure isomers or isomer mixtures of isophoronediamine, hexamethylene-1,6-diamine, bis(p-aminocyclohexyl)methane, 1,3- or 1,4-bis(aminomethyl)cyclohexane or isomer mixtures thereof, xylylenediamine or isomer mixtures thereof, pentane-1,5-diamine or 1,8-diamino-4-(aminomethyl)octane, preferably pentane-1,5-diamine.

4. The process as in either of clauses 1 and 2, wherein the diamines used are aromatic amines such as tolylene-2,4/2,6-diamine mixtures of isomer ratios 80/20 or 65/35 or the pure tolylene-2,4-diamine isomer, or isomer mixtures of diaminonaphthalene or of diaminodiphenylmethane.

5. The process as in any of clauses 1 to 4, wherein the amine stream and/or the phosgene stream is heated to 250° C. to 450° C. before being fed into the mixer.

6. The process as in any of clauses 1 to 5, wherein the phosgene is used in excess based on the amine and the molar phosgene excess based on an amino group is 30% to 300%, preferably 60% to 200%.

7. The process as in any of clauses 1 to 6, wherein the phosgene stream is introduced at a velocity of 15-90 m/s, more preferably 20-80 m/s, into the gaseous amine stream optionally diluted with an inert gas.

8. The process as in any of clauses 1 to 7, wherein the ratio of the length of the static mixer upstream of the plane of introduction to the length downstream of the plane of introduction is 0.1 to 1.0, preferably 0.2 to 0.8.

9. The process as in any of clauses 1 to 8, wherein the diameter of the mixing tube is such that the flow rate of the gas mixture of all components directly below (downstream of) the plane of introduction is 4-25 m/s, preferably 6-15 m/s, more preferably 8-12 m/s.

10. The process as in any of clauses 1 to 9, wherein the length of the static mixer of the KENICS type and the number of mixing elements therein is such that a degree of mixing of 50%-100%, preferably 80%-99%, more preferably 95%-98%, is attained at the end of the mixer.

11. The process as in any of clauses 1 to 10, wherein an inert gas is added to the amine and the number of mixing elements in the static mixer above the plane of introduction is such that the degree of mixing of amine and inert gas immediately upstream of the plane of introduction is 50% to 100%, preferably 70% to 95%.

12. The process as in any of clauses 1 to 11, wherein the temperature in the reaction space is 200° C. to 600° C., preferably 250° C. to 450° C.

13. The process as in any of clauses 1 to 12, wherein the pressure in the inlets to the reaction space is 200-3000 mbar abs., preferably 800-1500 mbar abs., and at the outlet from the reaction space is 150-2000 mbar abs., preferably 750-1440 mbar abs.

14. The process as in any of clauses 1 to 13, wherein a flow rate within the reaction space of 3 to 120 m/s, preferably 5 to 75 m/s, is maintained.

15. The process as in any of clauses 1 to 14, wherein the dwell time of the reaction mixture in the reactor is 0.05 s to 10 s, preferably 0.08 s to 4 s.

1. A process for preparing di- or triisocyanates of formula (I) or (II) 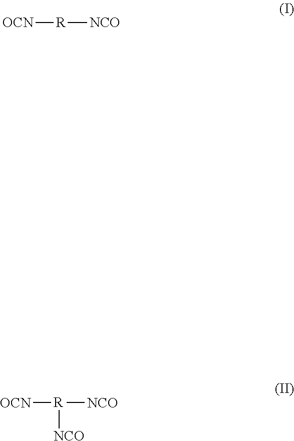
or mixtures of such di- or triisocyanates
wherein
R is a (cyclo)aliphatic, araliphatic or aromatic hydrocarbyl radical having up to 15 carbon atoms, with the proviso that at least 2 carbon atoms are arranged between the NCO groups,
by phosgenating corresponding di- or triamines of formula (III) or (IV) 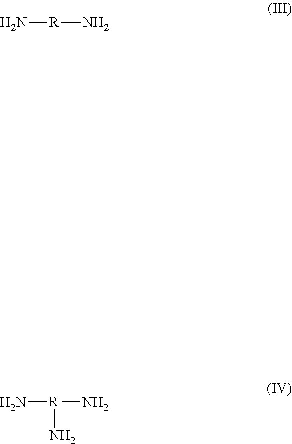
in which
R is as defined above,
in which the di- and triamines, optionally diluted with an inert gas or with the vapors of an inert solvent, and phosgene are heated separately to temperatures of 200° C. to 600° C., mixed in a tubular reactor and reacted, wherein
the phosgene is injected into the gaseous amine stream in the interior of a flow tube at an angle of ≦90° relative to the flow direction of the gaseous amine stream via an outer annular channel through a plurality of radial channels, with a static mixer of the KENICS type present in a coaxial arrangement in the interior of the flow tube.

2. The process according to claim 1, wherein the channels comprise holes and the angle is ≧60° and ≦90°.

3. The process according to claim 1, wherein the di- or triamines are aliphatic amines selected from the group consisting of isophoronediamine, hexamethylene-1,6-diamine, bis(p-aminocyclohexyl)methane, 1,3- or 1,4-bis(aminomethyl)cyclohexane and isomer mixtures thereof, xylylenediamine and isomer mixtures thereof, pentane-1,5-diamine and 1,8-diamino-4-(aminomethyl)octane.

4. The process according to claim 1, wherein the diamines are aromatic amines selected from the group consisting of tolylene-2,4/2,6-diamine mixtures of isomer ratios 80/20 or 65/35, pure tolylene-2,4-diamine isomer, isomer mixtures of diaminonaphthalene and isomer mixtures of diaminodiphenylmethane.

5. The process according to claim 1, wherein at least one of the amine stream and the phosgene stream is heated to 250° C. to 450° C. before being fed into the mixer.

6. The process according to claim 1, wherein the phosgene is used in excess based on the amine and the molar phosgene excess based on an amino group is 30% to 300%.

7. The process according to claim 1, wherein the phosgene stream is introduced at a velocity of 15-90 m/s into the gaseous amine stream optionally diluted with an inert gas.

8. The process according to claim 1, wherein the ratio of the length of the static mixer upstream of the plane of introduction to the length downstream of the plane of introduction is 0.1 to 1.0.

9. The process according to claim 1, wherein the diameter of the mixing tube is such that the flow rate of the gas mixture of all components directly below (downstream of) the plane of introduction is 4-25 m/s.

10. The process according to claim 1, wherein the length of the static mixer of the KENICS type and the number of mixing elements therein is such that a degree of mixing of 50%-100% is attained at the end of the mixer.

11. The process according to claim 1, wherein an inert gas is added to the amine and the number of mixing elements in the static mixer above the plane of introduction is such that the degree of mixing of amine and inert gas immediately upstream of the plane of introduction is 50% to 100%.

12. The process according to claim 1, wherein the temperature in the reaction space is 200° C. to 600° C.

13. The process according to claim 1, wherein the pressure in the inlets to the reaction space is 200-3000 mbar abs., and at the outlet from the reaction space is 150-2000 mbar abs.

14. The process according to claim 1, wherein a flow rate within the reaction space of 3 to 120 m/s is maintained.

15. The process according to claim 1, wherein the dwell time of the reaction mixture in the reactor is 0.05 s to 10 s.

16. The process according to claim 2, wherein the angle is ≧75° and ≦90°.

17. The process according to claim 2, wherein the angle is 90°.

18. The process according to claim 1, wherein the di- or triamine is pentane-1,5-diamine.

19. The process according to claim 1, wherein the length of the static mixer of the KENICS type and the number of mixing elements therein is such that a degree of mixing of 80%-99% is attained at the end of the mixer.

20. The process according to claim 1, wherein an inert gas is added to the amine and the number of mixing elements in the static mixer above the plane of introduction is such that the degree of mixing of amine and inert gas immediately upstream of the plane of introduction is 70% to 95%.
